(19) United States
(12) Patent Application Publication
(10) Pub. No.: US 2025/0212911 A1
(43) Pub. Date: Jul. 3, 2025

(54) PEA PROTEIN CONCENTRATES, AND USES THEREOF

(71) Applicant: BENSON HILL HOLDINGS, INC., St. Louis, MO (US)

(72) Inventors: Bala PUDOTA, St. Louis, MO (US); Keith PETROFSKY, St. Louis, MO (US)

(21) Appl. No.: 18/852,076

(22) PCT Filed: Mar. 30, 2023
(86) PCT No.: PCT/US2023/016980

§ 371 Date: Sep. 27, 2024

Related U.S. Application Data
(60) Provisional application No. 63/326,731, filed on Apr. 1, 2022.

(51) Int. Cl. A23J 1/14; A23L 11/00

(52) U.S. Cl. CPC A23J 1/14; A23L 11/05

(57) ABSTRACT

The present disclosure relates generally to pea products derived from high protein peas, and more specifically to protein-enriched pea compositions having high protein content and methods of preparing such pea compositions. The present disclosure also relates to uses of the protein-enriched pea compositions in food products.

### CROSS-REFERENCE TO RELATED APPLICATIONS

This application claims priority to U.S. Provisional Patent Application No. 63/326,731, filed Apr. 1, 2022, which is incorporated herein by reference in its entirety.

### FIELD

The present disclosure relates generally to pea products derived from high protein peas, and more specifically to protein-enriched pea compositions having high protein content and methods of preparing such compositions. The present disclosure also relates to uses of the protein-enriched pea compositions in food products.

### BACKGROUND

There is growing consumer interest in plant-based foods. Peas represent an attractive renewable source of plant-based protein for use in foodstuffs. However, the protein content of unprocessed peas is too low for many product applications. As a result, peas must be subjected to further processes in order to obtain compositions with increased protein content. Such processes may be divided into two categories: wet fractionation and dry fractionation.

Wet fractionation typically involves the exploitation of the solubility properties of different nutritional components in order to isolate protein-rich compositions from peas. This type of process typically involves immersing peas in aqueous solutions containing acids, bases, and/or other chemical processing agents. As a result, protein-enriched pea compositions prepared via a wet process contain chemical residues from the processing solution. Further, the use of acids, bases, and/or chemical processing agents in the wet process can result in loss of native functionality in the pea proteins in the processed pea composition.

Generally, dry processing is a chemical-free process that uses no water or chemical agents, and is more cost-effective than wet processing. Peas are milled, and the resulting particles are separated into protein- and starch-rich fractions on the basis of physical properties, such as density or size. However, dry processing is typically less efficient than wet processing, and it is generally difficult to obtain pea compositions with high protein content without also sustaining substantial losses to process yield. As a result, dry processing methods have not found significant use in the production of high-protein pea compositions on an economically viable scale.

### BRIEF SUMMARY

In one aspect, provided herein are methods of producing a protein-enriched pea composition. Such methods can produce the protein-enriched pea composition on commercially viable yields and scale, and thus addresses a need in the art. In some embodiments, the method comprises providing peas having a high protein content; grinding the peas to provide a ground pea composition; and isolating a protein-enriched pea composition from the ground pea composition, wherein the protein enriched pea composition comprises at least about 60% pea proteins on a dry weight basis.

In another aspect, provided herein are protein-enriched pea compositions obtainable by or produced according to the methods described herein.

In yet another aspect, provided herein are protein-enriched pea composition comprising at least about 60% pea protein by weight. In some embodiments, provided herein are protein-enriched pea compositions comprising a plurality of particles and at least about 60% pea protein by weight, wherein less than 90% of the particles are smaller than a given threshold size. In some variations, the threshold size is between about 15 μm and about 20 μm. In some embodiments, provided herein are protein-enriched pea compositions comprising a plurality of particles and at least about 60% pea protein by weight, wherein at least about 10 weight % of the particles in the protein-enriched pea composition have a particle size of at least about 20 μm. In some embodiments, provided herein are protein-enriched pea compositions comprising a plurality of particles and at least about 60% pea protein by weight, wherein less than 90% of the particles are smaller than a given threshold size, wherein said threshold size is between about 15 μm and about 20 μm, and at least about 10 weight % of the particles in the protein-enriched pea composition have a particle size of at least about 20 μm.

### DESCRIPTION OF THE FIGURES

The present application can be understood by reference to the following description taken in conjunction with the accompanying figures.

FIG. 1 depicts an exemplary plot showing the protein content of pea compositions relative to the protein contents of the peas from which the pea compositions are produced.

FIG. 2 depicts an exemplary process for preparing a protein-enriched pea composition

FIG. 3 depicts the protein content of whole peas, protein-rich fractions, and starch-rich fractions for several pea varieties, as well as the protein-rich fraction yields for the same pea varieties

FIG. 4 depicts the ash content of dehulled peas, protein-rich fractions, and starch-rich fractions for several pea varieties.

FIG. 5 depicts the damaged starch content of the protein-rich fraction and starch-rich fraction obtained for several pea varieties.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### I. High Protein Peas

In one aspect, provided herein are methods of producing protein-enriched pea compositions having a high protein content that are derived from peas having a high initial protein content, as well as methods of processing the same.

In some embodiments, pea varieties with a high protein content suitable for use in the methods described herein include, for example, field pea cultivars designated 3997496, 3997499, 3998902, 4004764, 4004781, and 4009968.

In some embodiments, the high protein peas comprise peas of field pea cultivar 3997496. In some embodiments, pea varieties with a high protein content suitable for use in the methods described herein are described in Table 1S. Additional description related to field pea cultivar 3997496 may be found in U.S. patent application Ser. No. 17/976,731, which is incorporated herein by reference in its entirety, including with respect to paragraphs [0042]-[0044] on pages 6-7 and paragraph [0232] on page 59.

TABLE 1S
Field Pea Cultivar 3997496 Variety Description Information

| Characteristic | 3997496 |
|---|---|
| Plant type | Yellow Field Pea |
| Plant habitat | Determinate |
| Plant height | Greater than 50 cm |
| Stem fascination | Absent |
| Presence of leaflets | Semi-leafless |
| Maturity | Medium |
| Flower color | White |
| Stipules | Semi-leafless |
| Time of flowering | Medium |
| Protein content on dry basis | 29.5% |
| Stipule development | Normal |
| Pod length (observed at first flowering | Medium |
| node) | |
| Pod width (observed at first flowering node) | Medium |
| Pod color (immature) | Light-green |
| Pod curvature (fully swollen) | Absent |
| Seed shape | Round |
| Weight (g/1000 seeds) | 250 |
| Color of cotyledon | Yellow |
| Black of hilum | Absent |
| Reaction to Powdery Mildew | Resistant |
| Seed size | Medium-large |
| Seed surface | Smooth |
| Color pattern | Monocolor |
| Hilum | White |
| Primary color | Yellow-green |

In some embodiments, the high protein peas comprise peas of field pea cultivar 3997499. In some embodiments, pea varieties with a high protein content suitable for use in the methods described herein are described in Table 2S. Additional description related to field pea cultivar 3997499 may be found in U.S. patent application Ser. No. 17/976,793, which is incorporated herein by reference in its entirety, including with respect to paragraphs [0042]-[0044] on pages 6-7 and paragraph [0232] on page 59.

TABLE 2S
Field Pea Cultivar 3997499 Variety Description Information

| Characteristic | 3997499 |
|---|---|
| Plant type | Yellow Field Pea |
| Plant habitat | Determinate |
| Plant height | Greater than 50 cm |
| Stem fascination | Absent |
| Presence of leaflets | Semi-leafless |
| Maturity | Medium |
| Flower color | White |
| Stipules | Semi-leafless |
| Time of flowering | Medium |
| Protein content on dry basis | 28.4% |
| Stipule development | Normal |
| Pod length (observed at first flowering | Medium |
| node) | |
| Pod width (observed at first flowering node) | Medium |
| Pod color (immature) | Light-green |
| Pod curvature (fully swollen) | Absent |
| Seed shape | Slightly Round |
| Weight (g/1000 seeds) | 230 |
| Color of cotyledon | Yellow |
| Black of hilum | Absent |
| Reaction to Powdery Mildew | Resistant |
| Seed size | Medium-large |
| Seed surface | Smooth |
| Color pattern | Monocolor |
| Hilum | White |
| Primary color | Yellow-green |

In some embodiments, the high protein peas comprise peas of field pea cultivar 3998902. In some embodiments, pea varieties with a high protein content suitable for use in the methods described herein are described in Table 3S. Additional description related to field pea cultivar 3998902 may be found in U.S. patent application Ser. No. 17/976,791, which is incorporated herein by reference in its entirety, including with respect to paragraphs [0042]-[0044] on pages 6-7 and paragraph [0232] on page 59.

TABLE 3S
Field Pea Cultivar 3998902 Variety Description Information

| Characteristic | 3998902 |
|---|---|
| Plant type | Yellow Field Pea |
| Plant habitat | Determinate |
| Plant height | Greater than 50 cm |
| Stem fascination | Absent |
| Presence of leaflets | Semi-leafless |
| Maturity | Medium |
| Flower color | White |
| Stipules | Semi-leafless |
| Time of flowering | Medium |
| Protein content on dry basis | 29.1% |
| Stipule development | Normal |
| Pod length (observed at first flowering | Medium |
| node) | |
| Pod width (observed at first flowering node) | Medium |
| Pod color (immature) | Light-green |
| Pod curvature (fully swollen) | Absent |
| Seed shape | Round |
| Weight (g/1000 seeds) | 215 |
| Color of cotyledon | Yellow |
| Black of hilum | Absent |
| Reaction to Powdery Mildew | Resistant |
| Seed size | Medium-large |
| Seed surface | Smooth |
| Color pattern | Monocolor |
| Hilum | White |
| Primary color | Yellow-green |

In some embodiments, the high protein peas comprise peas of field pea cultivar 4004764. In some embodiments, pea varieties with a high protein content suitable for use in the methods described herein are described in Table 4S. Additional description related to field pea cultivar 4004764 may be found in U.S. patent application Ser. No. 17/976,794, which is incorporated herein by reference in its entirety, including with respect to paragraphs [0042]-[0044] on pages 6-7, and paragraph [0232] on page 59.

TABLE 4S
Field Pea Cultivar 4004764 Variety Description Information

| Characteristic | 4004764 |
|---|---|
| Plant type | Yellow Field Pea |
| Plant habitat | Determinate |
| Plant height | Greater than 50 cm |
| Stem fascination | Absent |
| Presence of leaflets | Semi-leafless |
| Maturity | Medium |
| Flower color | White |
| Stipules | Semi-leafless |
| Time of flowering | Medium |
| Protein content on dry basis | 28.4% |
| Stipule development | Normal |
| Pod length (observed at first flowering | Medium |
| node) | |
| Pod width (observed at first flowering node) | Medium |
| Pod color (immature) | Light-green |
| Pod curvature (fully swollen) | Absent |
| Seed shape | Round |
| Weight (g/1000 seeds) | 232 |
| Color of cotyledon | Yellow |
| Black of hilum | Absent |
| Reaction to Powdery Mildew | Susceptible |
| Seed size | Medium-large |
| Seed surface | Smooth |
| Color pattern | Monocolor |
| Hilum | White |
| Primary color | Yellow-green |

In some embodiments, the high protein peas comprise peas of field pea cultivar 4004781. In some embodiments, pea varieties with a high protein content suitable for use in the methods described herein are described in Table 5S. Additional description related to field pea cultivar 4004781 may be found in U.S. patent application Ser. No. 17/976,736, which is incorporated herein by reference in its entirety, including with respect to paragraphs [0042]-[0044] on pages 6-7 and paragraph [0232] on page 59.

TABLE 5S
Field Pea Cultivar 4004781 Variety Description Information

| Characteristic | 4004781 |
|---|---|
| Plant type | Yellow Field Pea |
| Plant habitat | Determinate |
| Plant height | Greater than 50 cm |
| Stem fascination | Absent |
| Presence of leaflets | Semi-leafless |
| Maturity | Medium |
| Flower color | White |
| Stipules | Semi-leafless |
| Time of flowering | Medium |
| Protein content on dry basis | 28% |
| Stipule development | Normal |
| Pod length (observed at first flowering | Medium |
| node) | |
| Pod width (observed at first flowering node) | Medium |
| Pod color (immature) | Light-green |
| Pod curvature (fully swollen) | Absent |
| Seed shape | Round |
| Weight (g/1000 seeds) | 242 |
| Color of cotyledon | Yellow |
| Black of hilum | Absent |
| Reaction to Powdery Mildew | Susceptible |
| Seed size | Medium-large |
| Seed surface | Smooth |
| Color pattern | Monocolor |
| Hilum | White |
| Primary color | Yellow-green |

In some embodiments, the high protein peas comprise peas of field pea cultivar 4009968. In some embodiments, pea varieties with a high protein content suitable for use in the methods described herein are described in Table 6S. Additional description related to field pea cultivar 4009968 may be found in U.S. patent application Ser. No. 17/976,797, which is incorporated herein by reference in its entirety, including with respect to paragraphs [0042]-[0044] on pages 6-7 and paragraph [0232] on page 59.

TABLE 6S
Field Pea Cultivar 4009968 Variety Description Information

| Characteristic | 4009968 |
|---|---|
| Plant type | Yellow Field Pea |
| Plant habitat | Determinate |
| Plant height | Greater than 50 cm |
| Stem fascination | Absent |
| Presence of leaflets | Semi-leafless |
| Maturity | Medium |
| Flower color | White |
| Stipules | Semi-leafless |
| Time of flowering | Medium |
| Protein content on dry basis | 28.6% |
| Stipule development | Normal |
| Pod length (observed at first flowering | Medium |
| node) | |
| Pod width (observed at first flowering node) | Medium |
| Pod color (immature) | Light-green |
| Pod curvature (fully swollen) | Absent |
| Seed shape | Slightly Round |
| Weight (g/1000 seeds) | 238 |
| Color of cotyledon | Yellow |
| Black of hilum | Absent |
| Reaction to Powdery Mildew | Moderately susceptible |
| Seed size | Medium-large |
| Seed surface | Smooth |
| Color pattern | Monocolor |
| Hilum | White |
| Primary color | Yellow-green |

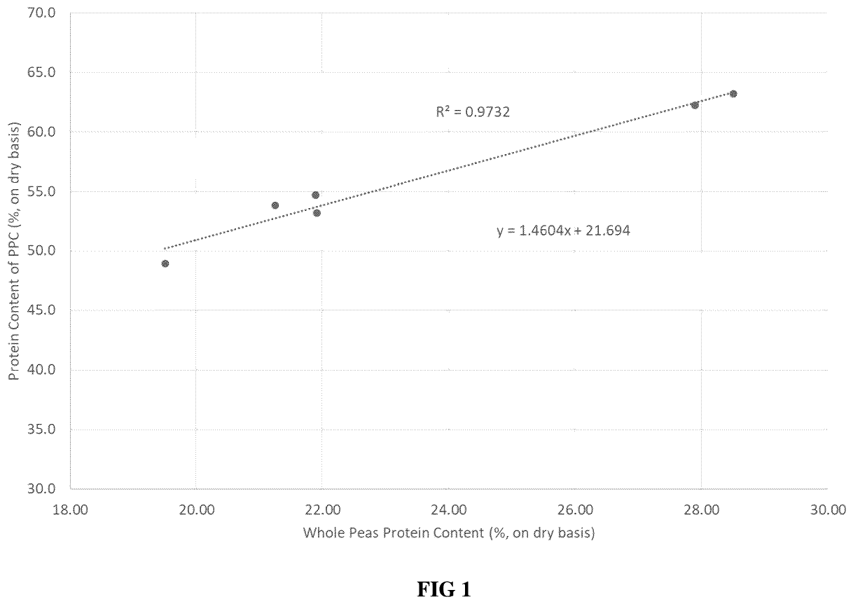

FIG. 1 illustrates an exemplary plot showing the protein content of pea compositions relative to the protein contents of the peas from which the pea compositions are produced. As shown in FIG. 1, the protein content in the pea compositions is directly proportional to the protein content initially present in the peas, and thus, the protein content of any single origin pea product is limited to the initial protein content of the source peas.

In some embodiments, the peas of the present disclosure comprise high protein contents, which lead to the production of protein-enriched pea compositions using the methods described in the present disclosure.

In some embodiments, the protein content of the peas having a high protein content may be described as the average protein content of the peas. In some embodiments, the protein content of the peas having a high protein content may be an average protein content of the peas for a given mass of peas (e.g., per 1 kilogram mass). In other embodiments, the protein content of the peas having a high protein content may be characterized by a distribution of protein contents.

In some embodiments, the peas having a high protein content may be characterized by protein content as determined by a combustion method (e.g., AOAC 990.03). In other embodiments, the peas having a high protein content may be characterized by protein content as determined by the Kjeldahl method (e.g., AOAC 928.08). In yet other embodiments, the peas having high protein content may be characterized by protein content as determined by near-infrared spectroscopy (NIRS).

In still other embodiments, the step of providing peas having a high protein content may be achieved by sorting and/or harvesting practices selective for peas having high protein content. For example, in some embodiments, the methods provided herein comprise separating a plurality of peas according to protein content to provide at least a portion of peas having a high protein content from the plurality of peas. In some embodiments, the separation of the plurality of peas according to protein content is a batch process. In some embodiments, the separation of the plurality of peas according to protein content is a continuous process. In certain embodiments, the separation of the plurality of peas according to protein content is a high-throughput process. In some embodiments, the separation of the plurality of peas according to protein content comprises determining protein content of the plurality of peas by NIRS. In some embodiments, the sorting of peas as described herein is performed manually. In other embodiments, the sorting of peas as described herein is performed mechanically.

In some variations of the foregoing embodiments, at least a portion of the peas used has a protein content of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40% pea protein on a dry weight basis. In certain variations, at least a portion of the peas used has a protein content of at least about 25% pea protein on a dry weight basis. In some variations, at least a portion of the peas used has a protein content of at least about 28% pea protein on a dry weight basis. In other variations, at least a portion of the peas used has a protein content of at least about 30% pea protein on a dry weight basis.

In some embodiments, at least a portion of the peas used has a protein content of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% pea protein on a dry weight basis. In some embodiments, at least a portion of the peas used has a protein content in a range between any two of the preceding enumerated values. In some embodiments, at least a portion of the peas used has a protein content of between about 20% and about 40%, between about 25% and about 40%, between about 26% and about 40%, between about 27% and about 40%, between about 28% and about 40%, between about 29% and about 40%, between about 30% and about 40%, between about 20% and about 35%, between about 25% and about 35%, between about 26% and about 35%, between about 27% and about 35%, between about 28% and about 35%, between about 29% and about 35%, between about 30% and about 35%, between about 20% and about 30%, or between about 25% and about 30% pea protein on a dry weight basis.

In other variations of the foregoing embodiments, the peas used have an average protein content of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40% pea protein on a dry weight basis. In certain variations, the peas used have an average protein content of at least about 25% pea protein on a dry weight basis. In some variations, the peas used have an average protein content of at least about 28% pea protein on a dry weight basis. In other variations, the peas used have an average protein content of at least about 30% pea protein on a dry weight basis.

In some embodiments, the peas used have an average protein content of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% pea protein on a dry weight basis. In some embodiments, the peas used have an average protein content in a range between any two of the preceding enumerated values. In some embodiments, the peas used have an average protein content of between about 20% and about 40%, between about 25% and about 40%, between about 26% and about 40%, between about 27% and about 40%, between about 28% and about 40%, between about 29% and about 40%, between about 30% and about 40%, between about 20% and about 35%, between about 25% and about 35%, between about 26% and about 35%, between about 27% and about 35%, between about 28% and about 35%, between about 29% and about 35%, between about 30% and about 35%, between about 20% and about 30%, or between about 25% and about 30% pea protein on a dry weight basis.

In yet other variations of the foregoing embodiments, the majority of the peas used have a protein content of at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40% pea protein on a dry weight basis. In certain variations, the majority of the peas used have a protein content of at least about 25% pea protein on a dry weight basis. In some variations, the majority of the peas used have a protein content of at least about 28% pea protein on a dry weight basis. In other variations, the majority of the peas used have a protein content of at least about 30% pea protein on a dry weight basis.

In some embodiments, the majority of the peas used have a protein content of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% pea protein on a dry weight basis. In some embodiments, the majority of the peas used have a protein content in a range between any two of the preceding enumerated values. In some embodiments, the majority of the peas used have a protein content of between about 20% and about 40%, between about 25% and about 40%, between about 26% and about 40%, between about 27% and about 40%, between about 28% and about 40%, between about 29% and about 40%, between about 30% and about 40%, between about 20% and about 35%, between about 25% and about 35%, between about 26% and about 35%, between about 27% and about 35%, between about 28% and about 35%, between about 29% and about 35%, between about 30% and about 35%, between about 20% and about 30%, or between about 25% and about 30% pea protein on a dry weight basis.

In some embodiments, the peas used have a high protein content and a low starch content. In some embodiments, the peas used have an average protein content of at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, or at least about 40% pea protein on a dry weight basis and an average starch content of less than about 45%, less than about 44%, less than about 43%, less than about 42%, less than about 41%, less than about 40%, less than about 39%, or less than about 38%, less than about 37%, less than about 36%, or less than about 35%. In some embodiments the peas used have an average protein content of at least about 30% and an average starch content of less than about 45% on a dry weight basis. In some embodiments, the peas used have a high protein content and a low starch content. In some embodiments, the peas used have an average protein content of between about 25%, and about 40% pea protein on a dry weight basis and an average starch content of between about 30% and about 45%.

In some embodiments, the peas used have an average protein to fiber ratio of at least about 0.55, at least about 0.6, at least about 0.65, at least about 0.7, at least about 0.75, at least about 0.8, or at least about 0.85. In some embodiments, the peas used have an average protein to fiber ratio of between about 0.55 and about 0.95, between about 0.6 and about 0.95, between about 0.65 and about 0.95, between about 0.7 and about 0.95, between about 0.75 and about 0.95, between about 0.8 and about 0.95, or between about 0.85 and about 0.95. In some embodiments, the peas used have an average protein to fiber ratio of about 0.55, about 0.6, about 0.65, about 0.7, about 0.75, about 0.8, or about 0.85. In some embodiments, the peas used have an average protein to fiber ratio of at least about 0.75. In some embodiments, the peas used have an average protein to fiber ratio of about 0.75.

In some embodiments, the peas used have a higher protein content and a lower starch content as compared to a commodity pea. In some embodiments, the peas used have a higher protein content and a lower starch content as compared to a commodity pea, while having a composition that is otherwise similar to that of a commodity pea. In some embodiments, the peas used have a higher protein content and a lower starch content as compared to a commodity pea, while having a similar average fat content, total dietary fiber content, and ash content as a commodity pea.

In some embodiments, the peas having a high protein content as used in the methods provided are distinguished from commodity peas. Commodity peas may have a protein content of less than 25%, or between about 20% and about 25%, on a dry weight basis. Similarly, the protein-enriched pea compositions obtained from the peas having a high protein content described herein are distinguished from pea protein products obtained from commodity peas, such as pea flours, pea protein concentrates, and pea protein isolates.

The percent composition of a given component in a pea or sample of peas may be described on an “as-is” basis or on a “dry-weight” basis. The percent composition of a given component may be converted between an “as-is” basis and a “dry-weight” basis using the following equation:

$$( protein content , “ dry - weight ” basis , \% ) = ( protein content , “ as - is ” basis , \% ) / ( ( 100 \% ) - ( moisture content , \% ) ) \qquad Eq . 1$$

The word “protein” in the above equation may be interchanged with any other pea component for which conversion between an “as-is” and a “dry-weight” basis is needed, including, e.g., starch. The moisture content of a pea or pea product may be determined using any suitable methods or techniques known in in the art. For example, methods known in the art for analyzing the composition of soybeans may also be used to analyze the content of peas (See e.g., Eys, J. E. Van. Manual of Quality Analyses For Soybean Products in The Feed Industry, 2^{nd} ed. U.S. Soybean Export Council).

The present invention is not limited to whether the peas comprise transgenic polynucleotides or proteins. The peas used in the Examples herein are non-transgenic and there are circumstances when using peas lacking transgenic traits, genome edits, or any other form of mutation (e.g., a change in a polynucleotide sequence) is necessary and/or beneficial. However, combining the teachings herein with a wide range of transgenic plants, or plants containing genome edits or any other form of mutation to confer new traits or combinations thereof is also envisioned.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, gRNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

In certain embodiments, peas as disclosed herein may be obtained from pea plants modified to exhibit at least one desired trait, and/or combinations thereof. The disclosed innovations are not limited to any set of traits that can be considered desirable, but nonlimiting examples include male sterility, herbicide tolerance, pest tolerance, disease tolerance, modified fatty acid metabolism, modified carbohydrate metabolism, modified seed yield, modified seed oil, modified seed protein, modified lodging resistance, modified shattering, modified iron-deficiency chlorosis, modified water use efficiency, and/or combinations thereof.

In certain embodiments, a user can combine the teachings herein with high-density molecular marker profiles spanning substantially the entire pea genome to estimate the value of selecting certain candidates in a breeding program in a process commonly known as genomic selection.

In some embodiments of the present disclosure, the peas having high protein content as utilized herein may be characterized by any suitable methods known in the art for genetic analysis. In some embodiments, the peas as utilized herein may be characterized by genetic analysis as comprising one or more genetic markers associated with high protein content.

### II. Processing

In one aspect, provided herein are methods of processing peas having high protein content (e.g., at least about 25%, at least about 26%, at least about 27%, or at least about 28% pea protein on a dry weight basis) to obtain protein-enriched pea compositions, such as protein-enriched pea ingredients, having a protein content of at least about 60% on a dry weight basis.

In one aspect, provided herein is a method of producing a protein-enriched pea composition comprising providing peas having a high protein content; grinding the peas to provide a ground pea composition; and isolating a protein-enriched pea composition from the ground pea composition from the ground pea composition, wherein the protein-enriched pea composition comprises at least about 60% pea proteins on a dry weight basis. In some variations of the foregoing embodiment, the peas having a high protein content comprise at least about 20%, at least about 21%, at least about 22%, at least about 23%, at least about 24%, at least about 25%, at least about 26%, at least about 27%, at least about 28%, at least about 29%, or at least about 30% pea protein on a dry weight basis. In certain variations the peas comprise at least about 25% pea protein on a dry weight basis. In some variations the peas comprise at least about 27% pea protein on a dry weight basis. In some variations the peas comprise at least about 28% pea protein on a dry weight basis.

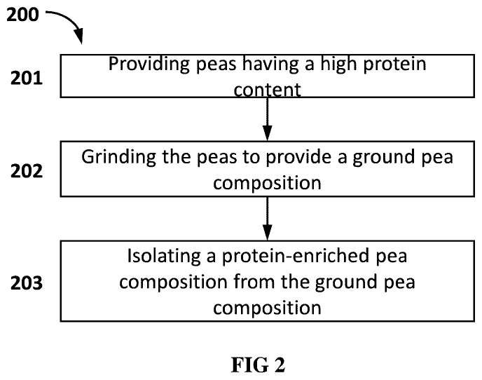

With reference to FIG. 2, process 200 is an exemplary process to prepare a protein-enriched pea composition. In step 201, peas having a high protein content are provided. In some variations, the peas may have a protein content as described in the preceding paragraph. In one variation, the peas comprise at least about 25%, at least about 26%, at least about 27%, or at least about 28% pea protein on a dry weight basis. In another variation, the peas comprise between about 25% and about 40%, between about 26% and about 40%, between about 27% and about 40%, or between about 28% and about 40% pea protein on a dry weight basis.

With reference again to FIG. 2, in step 202, the peas are ground to produce a ground pea composition. In step 203, a protein-enriched pea composition is isolated from the ground pea composition, wherein the protein enriched pea composition comprises at least about 60% pea proteins on a dry weight basis.

It should be understood that, in other variations, process 200 may include additional processing steps. In yet other variations, certain steps in process 200 may be omitted.

In some embodiments, step 201 further comprises cleaning the peas.

In some embodiments, step 201 further comprises dehulling the peas. Any suitable techniques known in the art to remove the hulls from the peas may be employed. For example, in some variations, the peas are subjected to abrasive force in order to remove the hulls. In certain variations, dehulling may be carried out with an attrition mill or an impeller, or mechanical equivalent. In one variation, the dehulling step does not utilize wet removal methods, such as blanching, alkaline and/or aqueous dissolution. In certain embodiments, the peas undergo thermal treatment prior to hull removal. In some variations, such thermal treatment is a dry dehulling process, which is distinguished from a wet dehulling process that may involve, for example, blanching. In certain embodiments, the peas subjected to the thermal treatment may be characterized by their moisture content. In some variations, the treated peas obtained from the thermal treatment have a moisture content of less than or equal to about 20% w/w, less than or equal to about 17% w/w, less than or equal to about 15% w/w, less than or equal to about 12% w/w, less than or equal to about 10% w/w, or less than or equal to about 8% w/w, without further drying. In other variations, the treated peas obtained from the thermal treatment have a moisture content of between about 5% w/w and about 20% w/w, between about 5% w/w and about 17% w/w, between about 5% w/w and about 15% w/w, between about 5% w/w and about 12% w/w, between about 5% w/w and about 10% w/w, between about 5% w/w and about 8% w/w, between about 8% w/w and about 20% w/w, between about 8% w/w and about 17% w/w, between about 8% w/w and about 15% w/w, between about 8% w/w and about 12% w/w, between about 8% w/w and about 10% w/w, between about 10% w/w and about 20% w/w, between 10% w/w and about 17% w/w, between about 10% w/w and about 15% w/w, between about 10% w/w and about 12% w/w, between about 12% w/w and about 20% w/w, between about 12% w/w and about 17% w/w, between about 12% w/w and about 15% w/w, between about 15% w/w and about 20% w/w, between about 15% w/w and about 17% w/w, or between about 17% w/w and about 20% w/w, without further drying. In yet other variations, the treated peas obtained from the thermal treatment may be dried in order to control the moisture content prior to grinding.

In some variations, removing the hulls from the peas results in a mixture comprising dehulled peas and detached hulls. As such, the method may further comprise separating the detached hulls from the dehulled peas. In some embodiments, the method further comprises separating the dehulled peas from the detached hulls by hand separation, by sieving or screening, or aerodynamic separation (i.e., weight classification by aspiration).

In some embodiments, step 201 further comprises sorting the peas. In some embodiments, the peas are sorted according to size. In some embodiments, the peas are sorted according to density. In some embodiments, the peas are sorted according to color.

In some embodiments, step 201 further comprises splitting the peas. Splitting may be accomplished using any suitable techniques known in the art.

With reference to step 202 of FIG. 2, any suitable methods of grinding known in the art may be employed. For example, grinding can be performed using equipment such as a hammer mill, FitzMill, or Quadromill.

In some embodiments, the grinding of step 202 yields a ground pea composition with a particular particle size distribution. Compositions comprising a plurality of particles, such as the ground pea composition of process 200, may be characterized by reference to the D(n) of the composition. D(n) is defined as the particle size for which n % of the particles in a composition are smaller than the D(n). For example, D(90) denotes the particle size for which 90% of the particles in a composition are smaller than D(90). Further, a D(n) of at least X μm means that n % of the particles in a composition are smaller than a threshold size that is X μm or larger. For example, a D(90) of at least 45 μm means that 90% of the particles in a composition are smaller than a threshold size, and said threshold size is either 45 μm or larger than 45 μm.

In some embodiments, at least 90% of the particles in the ground pea composition are smaller than about 45 μm, smaller than about 40 μm, smaller than about 35 μm, or smaller than about 30 μm. In some embodiments, the ground pea composition has a D(90) of at least about 45 μm, at least about 40 μm, at least about 35 μm, or at least about 30 μm. In some embodiments, at least 50% of the particles in the ground pea composition are smaller than about 20 μm, smaller than about 18 μm, smaller than about 16 μm, or smaller than about 15 μm. In some embodiments, the ground pea composition has a D(50) of at least about 20 μm, at least about 18 μm, at least about 16 μm, or at least about 15 μm.

In some embodiments, step 203 of FIG. 2 also produces a starch-enriched pea composition. In some embodiments, the isolation of a protein-enriched pea composition in step 203 is accomplished using an air classification process. Air classification may be performed using any suitable techniques or instruments known in the art. For example, in some embodiments, the air classification step is performed using a classifier mill, a classifier ATP, or a contraplex pin mill. In some embodiments air classification is performed at a rate of at least about 2 kg/hr, at least about 2.5 kg/hr, at least about 3 kg/hr, at least about 3.5 kg/hr, or at least about 4 kg/hr.

In some embodiments, the air classification process is only performed once.

In some embodiments, the protein-enriched pea composition of step 203 comprises at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, or at least about 70% pea protein on a dry weight basis. In some embodiments, the protein-enriched pea composition comprises at least about 58% pea protein on a dry weight basis. In some embodiments, the protein-enriched pea composition comprises at least about 60% pea protein on a dry weight basis. In some embodiments, the protein-enriched pea composition comprises at least about 62% pea protein on a dry weight basis.

In some embodiments, the protein-enriched pea composition of step 203 comprises a protein content of about 55%, about 56%, about 57%, about 58%, about 59%, about 60%, about 61%, about 62%, about 63%, about 64%, about 65%, about 66%, about 67%, about 68%, about 69%, or about 70% pea protein on a dry weight basis. In some embodiments, the protein-enriched pea composition of step 203 comprises a protein content in a range between any two of the preceding enumerated values. In some embodiments, the protein-enriched composition of step 203 comprises a protein content between about 50% and about 75%, between about 55% and about 75%, between about 60% and about 75%, between about 61% and about 75%, between about 62% and about 75%, between about 63% and about 75%, between about 64% and about 75%, between about 65% and about 75%, between about 66% and about 75%, between about 67% and about 75%, between about 68% and about 75%, between about 69% and about 75%, between about 50% and about 70%, between about 55% and about 70%, between about 60% and about 70%, between about 61% and about 70%, between about 62% and about 70%, between about 63% and about 70%, between about 64% and about 70%, between about 65% and about 70%, between about 66% and about 70%, between about 67% and about 70%, between about 68% and about 70%, between about 69% and about 70%, between about 50% and about 65%, between about 55% and about 65%, between about 60% and about 65%, between about 61% and about 65%, between about 62% and about 65%, between about 63% and about 65%, or between about 64% and about 65% pea protein on a dry weight basis.

In some embodiments, the protein-enriched pea composition of step 203 comprises a plurality of particles. In some embodiments, 90% of the particles of the protein-enriched pea composition are smaller than about 25 μm, smaller than about 24 μm, smaller than about 23 μm, smaller than about 22 μm, smaller than about 21 μm, smaller than about 20 μm, smaller than about 19 μm, smaller than about 18 μm, smaller than about 17 μm, smaller than about 16 μm, smaller than about 15 μm smaller than about 14 μm, smaller than about 13 μm, smaller than about 12 μm, smaller than about 11 μm, or smaller than about 10 μm. In some embodiments, the protein-enriched pea composition has a D(90) of at least about 25 μm, at least about 24 μm, at least about 23 μm, at least about 22 μm, at least about 21 μm, at least about 20 μm, at least about 19 μm, at least about 18 μm, at least about 17 μm, at least about 16 μm, at least about 15 μm, at least about 14 μm, at least about 13 μm, at least about 12 μm, at least about 11 μm, or at least about 10 μm. In some embodiments, at least about 8%, at least about 9%, at least about 10%, at least about 11%, or at least about 12% of the particles, by weight, in the protein-enriched pea composition have a particle size greater than about 20 μm. In some embodiments, the protein-enriched pea composition of step 203 has a D(90) of about 25 μm, about 24 μm, about 23 μm, about 22 μm, about 21 μm, about 20 μm, about 19 μm, about 18 μm, about 17 μm, about 16 μm, about 15 μm, about 14 μm, about 13 μm, about 12 μm, about 11 μm, or about 10 μm. In some embodiments, the protein-enriched pea composition of step 203 has a D(90) in a range between any two of the preceding enumerated values.

In some embodiments, the protein-enriched pea composition of step 203 comprises a plurality of particles. In some embodiments, at least about 10% of the particles of the protein-enriched pea composition are larger than about 25 μm, larger than about 24 μm, larger than about 23 μm, larger than about 22 μm, larger than about 21 μm, larger than about 20 μm, larger than about 19 μm, larger than about 18 μm, larger than about 17 μm, larger than about 16 μm, larger than about 15 μm larger than about 14 μm, larger than about 13 μm, larger than about 12 μm, larger than about 11 μm, or larger than about 10 μm.

In some embodiments, the starch-enriched pea composition of step 203 comprises a plurality of particles In some embodiments, 90% of the particles in the starch-enriched pea composition have a particle size smaller than about 35 μm, about 40 μm, about 45 μm, about 50 μm, or about 55 μm. In some embodiments the starch-enriched pea composition has a D(90) of at least about 35 μm, at least about 40 μm, at least about 45 μm, at least about 50 μm, or at least about 55 μm. In some embodiments, at least about 55%, at least about 60%, or at least about 65% of the particles, by weight, in the starch-enriched pea composition have a particle size greater than 20 μm.

In some embodiments, the weight of the protein-enriched pea composition of step 203 is at least about 30%, at least about 29%, at least about 28%, at least about 27%, at least about 26%, at least about 25%, at least about 24%, at least about 23%, at least about 22%, at least about 21%, at least about 20%, at least about 19%, at least about 18%, at least about 17%, at least about 16%, or at least about 15% of the weight of the ground pea composition from which it is isolated. In some embodiments, the weight of the protein-enriched pea composition of step 203 is between about 15% and about 40% of the weight of the ground pea composition from which it is isolated. In some embodiments, the weight percent of the protein-enriched pea composition of step 203 is about 30%, about 29%, about 28%, about 27%, about 26%, about 25%, about 24%, about 23%, about 22%, about 21%, about 20%, about 19%, about 18%, about 17%, about 16%, or about 15% of the weight of the ground pea composition from which it was isolated. In some embodiments, the weight percent of the protein-enriched pea composition of step 203 relative to the weight of the ground pea composition from which it was isolated is within a range between any two of the preceding enumerated values. In some embodiments, the protein content of the protein-enriched pea composition is at least about 62% and the weight of the protein-enriched pea composition is at least about 25% of the weight of the ground pea composition from which it was isolated.

In some embodiments, the protein-enriched pea composition has a protein content of at least about 62%, and a D(90) of at least about 20 μm. In some embodiments, the protein-enriched pea composition has a protein content of at least about 60%, and a D(90) of at least about 20 μm. In some embodiments, the protein-enriched pea composition has a protein content of at least about 58%, and a D(90) of at least about 20 μm. In some embodiments, the protein-enriched pea composition has a protein content of at least about 62%, and a D(90) of at least about 16 μm.

In some embodiments, the ratio of a) the weight % protein content of the protein-enriched pea composition of step 203 to b) the yield of the protein-enriched pea composition of step 203 is less than about 3. In some embodiments, the ratio of a) the weight % protein content of the protein-enriched pea composition of step 203 to b) the yield of the protein-enriched pea composition of step 203 is less than about 3 and the protein enriched pea composition has a protein content of at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, or at least about 70% pea protein on a dry weight basis. In some embodiments, the ratio of a) the weight % protein content of the protein-enriched pea composition of step 203 to b) the yield of the protein-enriched pea composition of step 203 is less than about 3 and the protein enriched pea composition has a protein content of at least about 60% pea protein on a dry weight basis. In some embodiments, the ratio of a) the weight % protein content of the protein-enriched pea composition of step 203 to b) the yield of the protein-enriched pea composition of step 203 is less than about 2.5. In some embodiments, the ratio of a) the weight % protein content of the protein-enriched pea composition of step 203 to b) the yield of the protein-enriched pea composition of step 203 is less than about 2.5 and the protein enriched pea composition has a protein content of at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, or at least about 70% pea protein on a dry weight basis. In some embodiments, the ratio of a) the weight % protein content of the protein-enriched pea composition of step 203 to b) the yield of the protein-enriched pea composition of step 203 is less than about 2.5 and the protein enriched pea composition has a protein content of at least about 60% pea protein on a dry weight basis.

In some embodiments, a) the weight % protein content of the protein-enriched pea composition of step 203, multiplied by b) the % yield of the protein-enriched pea composition of step 203, is at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%. In some embodiments, a) the weight % protein content of the protein-enriched pea composition of step 203, multiplied by b) the % yield of the protein-enriched pea composition of step 203, is at least 14%. In some embodiments, a) the weight % protein content of the protein-enriched pea composition of step 203, multiplied by b) the % yield of the protein-enriched pea composition of step 203, is at least 17%. In some embodiments, a) the weight % protein content of the protein-enriched pea composition of step 203, multiplied by b) the % yield of the protein-enriched pea composition of step 203, is about 10%, about 11%, about 12%, about 13%, about 14%, about 15%, about 16%, about 17%, about 18%, about 19%, about 20%, about 21%, about 22%, about 23%, about 24%, or about 25%. In some embodiments, a) the weight % protein content of the protein-enriched pea composition of step 203, multiplied by b) the % yield of the protein-enriched pea composition of step 203, is in a range between any two of the values enumerated above.

In some embodiments, the protein-enriched pea composition of step 203 comprises between about 1% and 5% by weight crude fiber. In some embodiments, the protein-enriched pea composition of step 203 comprises between about 5% and 10% by weight ash. In some embodiments, the protein-enriched pea composition of step 203 comprises between about 1% and 5% by weight crude fiber and between about 5% and 10% by weight ash.

In some embodiments, the starch-enriched pea composition of step 203 comprises between about 10% and 30% by weight protein. In some embodiments, the starch-enriched pea composition of step 203 comprises between about 0.1% and 5% by weight crude fiber. In some embodiments, the starch-enriched pea composition of step 203 comprises between about 1% and 5% by weight ash. In some embodiments, the starch-enriched pea composition of step 203 comprises between about 1% and 5% by weight damaged starch.

Compared to conventional wet extraction methods, air classification is a chemical-free method of preparing protein-enriched pea compositions. Accordingly, in some embodiments, the protein-enriched pea compositions prepared according to process 200 possess more desirable chemical or physical properties relative to protein-enriched compositions prepared using a wet extraction method. In some embodiments, the protein-enriched pea composition of step 203 has a higher protein dispersibility index than that of pea compositions prepared using a wet extraction method. In some embodiments, the protein-enriched pea composition of step 203 has a protein dispersibility index of at least about 10%, at least about 20%, at least about 30%, at least about 40%, at least about 50%, at least about 60%, at least about 70%, at least about 75%, at least about 80%, at least about 90%, or about 100%. In some embodiments, the protein-enriched composition of step 203 is substantially free of any chemicals exogenous to the peas of step 201. In some embodiments, the protein-enriched composition of step 203 is substantially free of any hydrochloric acid or sodium hydroxide.

### III. Compositions

In addition to the methods of processing peas having high protein content (e.g. at least about 25% pea proteins of a dry weight basis, at least about 27% pea proteins of a dry weight basis, or at least about 28% pea proteins of a dry weight basis) provided herein, the present disclosure also provides protein-enriched pea compositions.

As detailed above the protein-enriched pea compositions are obtained from peas having high protein content as utilized herein. As detailed above, the peas having high protein content may be characterized by any suitable methods known in the art as having one or more genetic markers associated with the high protein content present in the peas.

Although the compositions of the protein-enriched pea compositions will differ from the source peas as a result of the processing methods described herein, the resulting protein-enriched pea compositions may contain detectable remnants of genetic material associated with the source peas having high protein content. In certain embodiments, the pea products provided herein may contain genetic material tied to particular genetic markers (e.g., biomarkers associated with high protein content) which were present in the source peas, and may be similarly characterized by genetic analysis to identify the same markers. In some embodiments, the protein-enriched pea compositions provided herein and obtained from peas as utilized herein may be characterized by genetic analysis. In some embodiments, any suitable techniques known in the art may be used to characterize the protein-enriched pea compositions provided herein as comprising one or more genetic markers associated with high protein content in peas. In some embodiments, the peas as utilized herein may be characterized by genetic analysis as comprising one or more genetic markers associated with high protein content.

The protein-enriched pea composition provided herein has a high pea protein content, including relative to the pea protein content of the peas from which the protein-enriched pea composition was obtained.

In some embodiments, the protein-enriched pea composition provided herein comprises at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, or at least about 70% pea protein on a dry weight basis. In some embodiments, the protein-enriched pea composition comprises at least about 58% pea protein on a dry weight basis. In some embodiments, the protein-enriched pea composition comprises at least about 60% pea protein on a dry weight basis. In some embodiments, the protein-enriched pea composition comprises at least about 62% pea protein on a dry weight basis.

In some embodiments, the protein-enriched pea composition provided herein comprises a protein content of about 55%, about 56%, about 57%, about 58%, about 59%, about 60%, about 61%, about 62%, about 63%, about 64%, about 65%, about 66%, about 67%, about 68%, about 69%, or about 70% pea protein on a dry weight basis. In some embodiments, the protein-enriched pea composition provided herein comprises a protein content in a range between any two of the preceding enumerated values. In some embodiments, the protein-enriched composition of step 203 comprises a protein content between about 50% and about 75%, between about 55% and about 75%, between about 60% and about 75%, between about 61% and about 75%, between about 62% and about 75%, between about 63% and about 75%, between about 64% and about 75%, between about 65% and about 75%, between about 66% and about 75%, between about 67% and about 75%, between about 68% and about 75%, between about 69% and about 75%, between about 50% and about 70%, between about 55% and about 70%, between about 60% and about 70%, between about 61% and about 70%, between about 62% and about 70%, between about 63% and about 70%, between about 64% and about 70%, between about 65% and about 70%, between about 66% and about 70%, between about 67% and about 70%, between about 68% and about 70%, between about 69% and about 70%, between about 50% and about 65%, between about 55% and about 65%, between about 60% and about 65%, between about 61% and about 65%, between about 62% and about 65%, between about 63% and about 65%, or between about 64% and about 65% pea protein on a dry weight basis.

In some embodiments, the protein-enriched pea composition provided herein comprises a plurality of particles. In some embodiments, 90% of the particles of the protein-enriched pea composition are smaller than about 25 μm, smaller than about 24 μm, smaller than about 23 μm, smaller than about 22 μm, smaller than about 21 μm, smaller than about 20 μm, smaller than about 19 μm, smaller than about 18 μm, smaller than about 17 μm, smaller than about 16 μm, smaller than about 15 μm smaller than about 14 μm, smaller than about 13 μm, smaller than about 12 μm, smaller than about 11 μm, or smaller than about 10 μm. In some embodiments, the protein-enriched pea composition has a D(90) of at least about 25 μm, at least about 24 μm, at least about 23 μm, at least about 22 μm, at least about 21 μm, at least about 20 μm, at least about 19 μm, at least about 18 μm, at least about 17 μm, at least about 16 μm, at least about 15 μm, at least about 14 μm, at least about 13 μm, at least about 12 μm, at least about 11 μm, or at least about 10 μm. In some embodiments, at least about 8%, at least about 9%, at least about 10%, at least about 11%, or at least about 12% of the particles, by weight, in the protein-enriched pea composition have a particle size greater than about 20 μm. In some embodiments, the protein-enriched pea composition provided herein has a D(90) of about 25 μm, about 24 μm, about 23 μm, about 22 μm, about 21 μm, about 20 μm, about 19 μm, about 18 μm, about 17 μm, about 16 μm, about 15 μm, about 14 μm, about 13 μm, about 12 μm, about 11 μm, or about 10 μm. In some embodiments, the protein-enriched pea composition provided herein has a D(90) in a range between any two of the preceding enumerated values.

In some embodiments, the protein-enriched pea composition provided herein comprises a plurality of particles. In some embodiments, at least about 10% of the particles of the protein-enriched pea composition are larger than about 25 μm, larger than about 24 μm, larger than about 23 μm, larger than about 22 μm, larger than about 21 μm, larger than about 20 μm, larger than about 19 μm, larger than about 18 μm, larger than about 17 μm, larger than about 16 μm, larger than about 15 μm larger than about 14 μm, larger than about 13 μm, larger than about 12 μm, larger than about 11 μm, or larger than about 10 μm.

In some embodiments, the protein-enriched pea composition has at least about 1.3 times, at least about 1.4 times, at least about 1.5 times, or at least about 1.6 times more protein content per unit weight than the peas from which the protein-enriched pea composition was obtained.

In some embodiments, the protein-enriched pea composition provided herein may be characterized by its crude fiber content. In some embodiments, the protein-enriched pea composition provided herein comprises between about 1% and 5% by weight crude fiber.

In some embodiments, the protein-enriched pea composition provided herein may be characterized by its ash content. In some embodiments, the protein-enriched pea composition provided herein comprises between about 5% and 10% by weight ash.

Compared to conventional wet extraction methods, air classification is a chemical-free method of preparing protein-enriched pea compositions. Accordingly, in some embodiments, the protein-enriched pea compositions provided herein possess more desirable chemical or physical properties relative to protein-enriched compositions prepared using a wet extraction method. In some embodiments, the protein-enriched pea composition provided herein has a higher protein dispersibility index than that of pea compositions prepared using a wet extraction method. In some embodiments, the protein-enriched pea composition provided herein has a protein dispersibility index of at least about 10%, at least about 20%, at least about 30%, at least about 40%, at least about 50%, at least about 60%, at least about 70%, at least about 75%, at least about 80%, at least about 90%, or about 100%. PDI may be measured using any suitable techniques known in the art. For example, in one variation, PDI is measured in accordance with the protocol set forth in Example 3, below. In some embodiments, the protein-enriched composition provided herein is substantially free of any chemicals exogenous to the peas from which the protein-enriched pea composition is obtained. In some embodiments, the protein-enriched composition provided herein is substantially free of any hydrochloric acid or sodium hydroxide.

In some embodiments, the protein-enriched composition provided herein comprises a plurality of particles. In some embodiments, 90% of the particles of the protein-enriched pea composition are smaller than about 21 μm, smaller than about 20 μm, smaller than about 19 μm, smaller than about 18 μm, smaller than about 17 μm, smaller than about 16 μm, or smaller than about 15 μm. In some embodiments, the protein-enriched pea composition has a D(90) of at least about 21 μm, at least about 20 μm, at least about 19 μm, at least about 18 μm, at least about 17 μm, at least about 16 μm, or at least about 15 μm. In some embodiments, at least about 8%, at least about 9%, at least about 10% or at least about 11% of the particles, by weight, in the protein-enriched pea composition have a particle size greater than about 20 μm.

In some embodiments, the protein-enriched pea composition has a protein content of at least about 62%, and a D(90) of at least about 20 μm. In some embodiments, the protein-enriched pea composition has a protein content of at least about 60%, and a D(90) of at least about 20 μm. In some embodiments, the protein-enriched pea composition has a protein content of at least about 58%, and a D(90) of at least about 20 μm. In some embodiments, the protein-enriched pea composition has a protein content of at least about 62%, and a D(90) of at least about 16 μm.

In some embodiments, the protein-enriched pea composition has a protein content of at least about 60%, and a D(90) of at least about 25 μm, about 24 μm, about 23 μm, about 22 μm, about 21 μm, about 20 μm, about 19 μm, about 18 μm, about 17 μm, about 16 μm, about 15 μm, about 14 μm, about 13 μm, about 12 μm, about 11 μm, or about 10 μm. In some embodiments, the protein-enriched pea composition has a protein content of at least about 62%, and a D(90) of at least about 25 μm, about 24 μm, about 23 μm, about 22 μm, about 21 μm, about 20 μm, about 19 μm, about 18 μm, about 17 μm, about 16 μm, about 15 μm, about 14 μm, about 13 μm, about 12 μm, about 11 μm, or about 10 μm.

In some embodiments, the protein-enriched pea composition has a protein content of at least about 60% and at least about 10% of the particles of the protein-enriched pea composition are larger than about 25 μm, larger than about 24 μm, larger than about 23 μm, larger than about 22 μm, larger than about 21 μm, larger than about 20 μm, larger than about 19 μm, larger than about 18 μm, larger than about 17 μm, larger than about 16 μm, larger than about 15 μm larger than about 14 μm, larger than about 13 μm, larger than about 12 μm, larger than about 11 μm, or larger than about 10 μm. In some embodiments, the protein-enriched pea composition has a protein content of at least about 62% and at least about 10% of the particles of the protein-enriched pea composition are larger than about 25 μm, larger than about 24 μm, larger than about 23 μm, larger than about 22 μm, larger than about 21 μm, larger than about 20 μm, larger than about 19 μm, larger than about 18 μm, larger than about 17 μm, larger than about 16 μm, larger than about 15 μm larger than about 14 μm, larger than about 13 μm, larger than about 12 μm, larger than about 11 μm, or larger than about 10 μm

In some embodiments, the protein-enriched composition provided herein is a flour.

The protein-enriched pea compositions provided herein may be used as ingredients in various food applications. With respect to certain applications, the protein-enriched pea compositions provided herein may demonstrate comparable and, in certain circumstances, superior properties as compared to other pea ingredients or pea compositions in the market, and thus may be advantageously incorporated into specific food products over competitor pea products. Exemplary products may include for example beverage products such as ready-to-drink beverages, or protein shake powders, dairy product substitutes including plant-based yogurt, cheese or milks, meat substitute products such as plant-based burgers, and egg substitutes.

In some variations of the foregoing, the food or beverage products have at least about 1, at least about 2, at least about 3, at least about 4, at least about 5, at least about 6, at least about 7, at least about 8, at least about 9, at least about 10 grams, at least about 12 g, at least about 15 g, or at least about 17 g of pea protein per serving. In some variations, the food or beverage products have between about 1 g and about 20 g of pea protein per serving. In certain variations, at least about 10%, at least about 20%, at least about 25%, at least about 30%, at least about 40%, at least about 50%, at least about 60%, at least about 70%, at least about 75%, at least about 80%, at least about 90%, or at least about 95% by weight of the protein in the food or beverage product is from pea protein. In some variations, between about 10% and about 100% of the protein in the food or beverage product is from pea protein.

The food and beverages products can include various other components other than the protein-enriched pea compositions described herein. For example, the food and beverage products may include, for example, water, flour, fats and oils, sweeteners (such as sugar), salt, leavening agents, fruit and vegetable juices, thickeners (such as pectin and other hydro colloids), anti-foaming agents, natural and artificial flavorings, preservatives, and coloring agents.

In another aspect, provided is a method of preparing food and/or beverages products. Such methods may include one or more of mixing/blending, pasteurizing and/or sterilizing, baking, fermenting, carbonating, leavening, and packaging.

In other aspects, the protein-enriched pea compositions herein may be used as or incorporated into pharmaceutical products. In certain variations of the foregoing aspect, the protein-enriched pea compositions have a pharmaceutical-grade purity. In other variations, the protein-enriched pea compositions have a protein purity of greater than or equal to about 99%.

In other aspects, the protein-enriched pea compositions herein may be used as or incorporated into dietary supplement products. In certain variations of the foregoing aspect, the protein-enriched pea compositions have a dietary supplement-grade purity. In other variations, the protein-enriched pea compositions have a protein purity of greater than or equal to about 99%.

In other aspects, the protein-enriched pea compositions herein may be used as or incorporated into cosmetic products. In certain variations of the foregoing aspect, the protein-enriched pea compositions have a cosmetic-grade purity. In other variations, the protein-enriched pea compositions have a protein purity of greater than or equal to about 99%.

In other aspects, the protein-enriched pea compositions herein may be used as or incorporated into medical foods. In certain variations of the foregoing aspect, the protein-enriched pea compositions have a medical food-grade purity. In other variations, the protein-enriched pea compositions have a protein purity of greater than or equal to about 99%.

In other aspects, the protein-enriched pea compositions herein may be used as or incorporated into infant formula products. In certain variations of the foregoing aspect, the protein-enriched pea compositions have an infant formula-grade purity. In other variations, the protein-enriched pea compositions have a protein purity of greater than or equal to about 99%.

### ENUMERATED EMBODIMENTS

The following enumerated embodiments are representative of some aspects of the invention.
1. A method of producing a protein-enriched pea composition, the method comprising:
   a) providing peas, wherein at least a portion of the peas has a protein content of at least about 25% pea proteins on a dry weight basis;
   b) grinding the peas to provide a ground pea composition; and
   c) isolating a protein-enriched pea composition from the ground pea composition, wherein the protein-enriched pea composition comprises at least about 60% pea proteins on a dry weight basis.
2. The method of embodiment 1, wherein step c) further produces a starch-enriched pea composition.
3. The method of any one of embodiment 1 or 2, further comprising dehulling the peas prior to the grinding step.
4. The method of any one of embodiments 1-3, wherein the isolation of a protein-enriched pea composition in step c) is accomplished using an air classification process.
5. The method of any one of embodiments 1-4, wherein the weight of the protein-enriched pea composition is at least about 23% of the weight of the ground pea composition.
6. The method of any one of embodiments 1-5, wherein a) the weight % protein content of the protein-enriched pea composition, multiplied by b) the % yield of the protein-enriched pea composition, is at least 14%.
7. The method of any one of embodiments 1-6, wherein the protein-enriched pea composition comprises one or more of the following:
   (i) between about 1% and 5% by weight crude fiber; and
   (ii) between about 5% and 10% by weight ash.
8. The method of any one of embodiments 2-7, wherein the starch-enriched pea composition comprises one or more of (i) to (iv):
   (i) between about 10% and 25% by weight protein;
   (ii) between about 0.1% and 5% by weight crude fiber;
   (iii) between about 1% and 5% by weight ash; and
   (iv) between about 1% and 5% by weight damaged starch.
9. The method of any one of embodiments 1-8, wherein the protein-enriched pea composition has a protein dispersibility index of at least 75.
10. The method of any one of embodiments 1-9, wherein a single separation step is performed in the method.
11. A protein-enriched pea composition produced according to the method of any one of embodiments 1-10.
12. The composition of embodiment 11, further comprising a protein dispersibility index of at least about 75.
13. The composition of embodiment 11 or 12, wherein the composition is a flour.

### EXAMPLES

The presently disclosed subject matter will be better understood by reference to the following Examples, which are provided as exemplary of the invention, and not by way of limitation.

### Example 1: Trial #1, Preparation of Protein-Enriched Pea Composition on a 100 lb Scale and Comparison with Commercial Pea Protein Compositions

Protein-enriched pea compositions were prepared from high-protein peas having an average starting protein content of 27.9% by weight using an air classification process. Three different compositions were prepared, with each composition corresponding to a different grind coarseness employed during processing. As a comparison, three additional pea protein compositions were prepared from peas of commodity variety #1 having an average starting protein content of 21.9%, using the same process and similar grind sizes.

Briefly, for each batch, 100 lb of peas were first dehulled. The dehulled peas were charged to a screw feeder, which was used to feed the dehulled peas into a classification mill at a predetermined rate. The classification mill was used to grind the dehulled peas into pea flour of a specified coarseness. For each pea variety, flours with D(90) values of about 35, 40, and 45 μm were prepared.

Next, an air classification process was used to separate each flour into a protein-rich fraction, and a starch-rich fraction. Flour collected from the grinding process was again charged into a screw feeder, which was used to feed the flour into an air classifier at a predetermined rate. The air classifier separated the flour into fine and course particles. The fine particles were pneumatically conveyed to a dust collector where the product was separated from air. Coarse particles were collected under the air classifier. A fan downstream of the dust collector supplies the pneumatic air for the system.

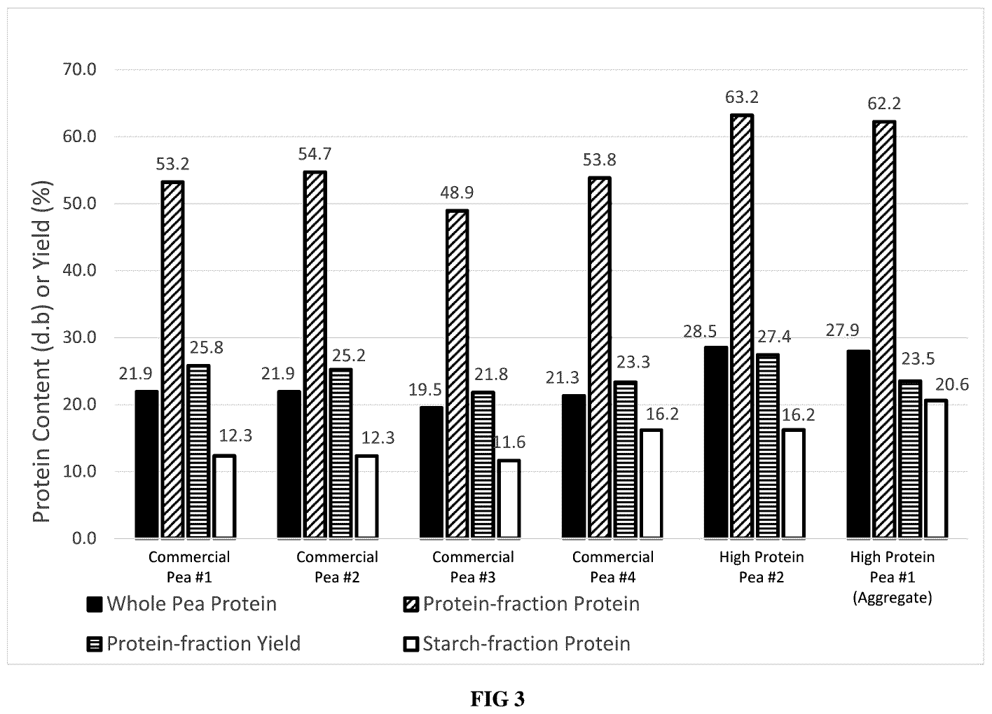

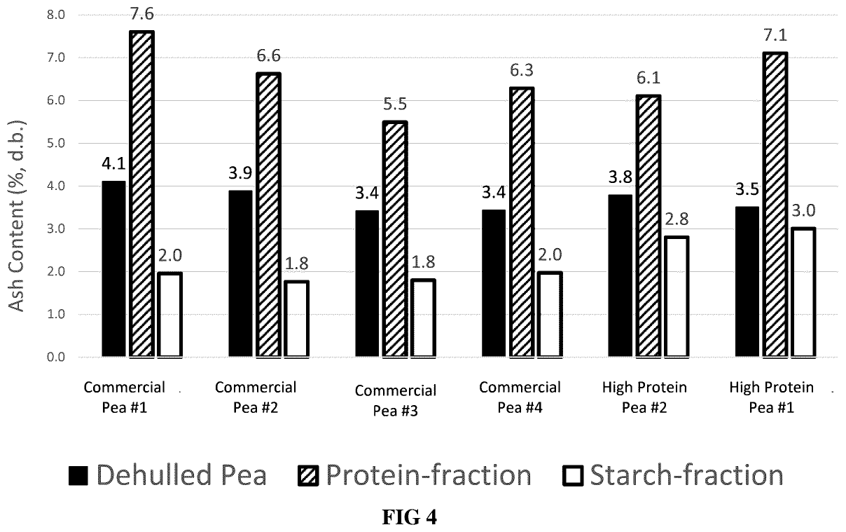

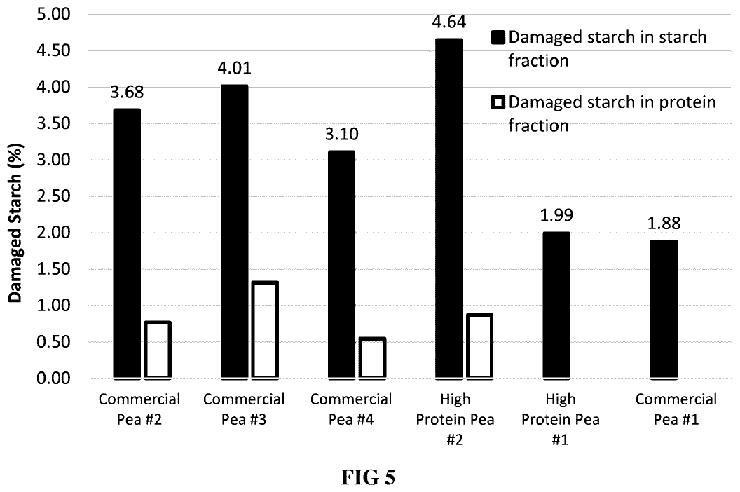

Using the process described above, protein-rich fractions comprising 60-62% protein by weight were obtained in 22-25% yield when the high protein pea compositions were used as the starting material, while protein-rich fractions comprising 52-54% protein by weight were obtained in 20-30% yield when peas of commodity variety #1 were used as the starting material. The chemical composition, particle size, and yields for each trial are summarized in table 1 and table 2, below. The composition-related information is also summarized in FIG. 3-5.

TABLE 1
Compositions of pea compositions prepared in 100 lb scale trials

| | | Dehulled Peas | | | | | | | | | | |
|---|---|---|---|---|---|---|---|---|---|---|---|---|
| | | | | | Flour | | | | | | | |
| | | | | | particle | Protein-rich Fraction | | | Starch-rich Fraction | | | |
| Whole Peas | | | Crude | | size | | Crude | | | Crude | | Damaged |
| | Protein | Protein | Fiber | Ash | D(90) | Protein | Fiber | Ash | Protein | Fiber | Ash | Starch |
| Variety | (%) | (%) | (%) | (%) | (μm) | (%) | (%) | (%) | (%) | (%) | (%) | (%) |
| High | 27.9 | 30.5 | 1.6 | 3.5 | 34 | 60.9 | 2.3 | 7.1 | 19.4 | 1.3 | 2.7 | 2.2 |
| Protein | | | | | 40 | 62.2 | 2.1 | 7.1 | 20.6 | 1.2 | 2.8 | 2.0 |
| Pea #1 | | | | | 43 | 60.2 | 2.3 | 7.0 | 19.8 | 1.2 | 2.7 | 2.0 |
| Commodity | 21.9 | 24.3 | 2.2 | 4.1 | 36 | 52.4 | 2.6 | 7.6 | 14.9 | 1.8 | 2.9 | 1.9 |
| Pea #1 | | | | | 41 | 53.2 | 2.3 | 7.6 | 16.2 | 1.6 | 3.0 | 1.9 |
| | | | | | 45 | 53.5 | 2.2 | 7.8 | 16.4 | 1.7 | 3.2 | 1.8 |

TABLE 2
Particle size and yield from 3 grind settings for peas prepared in 100 lb scale trials

| | | | | | | | | | Air Classification | | | | | |
|---|---|---|---|---|---|---|---|---|---|---|---|---|---|---|
| | | | | | | | | | Protein-rich | | | Starch-rich | | |
| Dehulled Peas | | | | | | Grinding | | | Fraction | | | Fraction | | |
| | Tapped | | | | | Pea Flour | | | | Over | | | Over | |
| | bulk | On screen (%) | | | | | | Over | | 20 | | | 20 | |
| | density | US. | US. | US. | Moisture | d(50) | d(90) | 20 μm | d(90) | μm | Yield | d(90) | μm | Yield |
| Variety | (#/ct.ft.) | #4 | #7 | #10 | (%) | (μm) | (μm) | (%) | (μm) | (%) | (%) | (μm) | (%) | (%) |
| High | 55.3 | 68.8 | 30.0 | 1.2 | 8.8 | 14.7 | 34.2 | 35.7 | 19.7 | 9.6 | 24.9 | 39.8 | 59.0 | 75.1 |
| Protein | | | | | | | | | | | | | | |
| Pea #1 | | | | | | 18.5 | 39.7 | 45.7 | 20.4 | 10.6 | 22.1- | 50.0 | 66.4 | 75.7- |
| | | | | | | | | | | | 24.8 | | | 77.9 |
| | | | | | | 18.6 | 42.5 | 46.1 | 20.6 | 10.8 | 24.9 | 45.3 | 65.3 | 75.1 |
| Commodity | 54.5 | 48.6 | 50.8 | 0.6 | 8.8 | 17.0 | 35.8 | 40.8 | 19.0 | 8.7 | 21.9- | 40.5 | 57.9 | 68.3- |
| Pea #1 | | | | | | | | | | | 31.6 | | | 78.0 |
| | | | | | | 18.3 | 40.6 | 44.9 | 18.9 | 8.5 | 24.5- | 47.4 | 61.9 | 73.1- |
| | | | | | | | | | | | 27.0 | | | 75.6 |
| | | | | | | 19.0 | 45.4 | 47.3 | 18.8 | 8.4 | 19.5- | 55.7 | 64.3 | 69.8- |
| | | | | | | | | | | | 30.1 | | | 80.4 |

### Example 2: Trial #2, Preparation of Protein-Enriched Pea Composition on a 5-10 kg Scale and Comparison with Commercial Pea Protein Compositions

Protein-enriched pea compositions were prepared from high-protein peas having an average starting protein content of 28.5% by weight using an air classification process. As a comparison, three additional pea protein compositions were prepared from peas of commodity varieties #2, #3, and #4 having an average starting protein contents of 21.9, 19.5, and 21.3% protein content, respectively, using the same process. A single grind setting was used in the preparation of all 4 compositions.

Briefly, for each batch, 5-10 kg of peas were first dehulled. Next, the dehulled peas were ground into a flour using a classifier mill. Each flour had a D(90) value of around 31-32 μm. Additional details regarding the particle size distribution for flours prepared from each pea variety may be found in table 3. Next, an air classification process was used to separate each flour into a protein-rich fine fraction, and a starch-rich coarse fraction. A feed rate of about 3 kg/hr was used when processing each flour. The particle size distributions of both fractions for each variety are summarized in table 4. Details related to the chemical composition of the protein-rich fine fractions obtained for each variety are summarized in FIG. 3-5.

TABLE 3
Particle size distribution of ground pea flour
prepared from 4 different pea varieties

| | Protein | | | |
|---|---|---|---|---|
| | Content of | D(50), | D(90), | D(4, 3), |
| Variety | Whole Peas | μm | μm | μm |
| Commodity Pea #2 | 21.9 | 16.4 | 32.0 | 17.3 |
| Commodity Pea #3 | 19.5 | 16.1 | 31.5 | 18.6 |
| Commodity Pea #4 | 21.3 | 15.4 | 30.8 | 20.7 |
| High Protein Pea | 28.5 | 14.8 | 31.2 | 29.2 |
| #2 | | | | |

TABLE 4
Particle size distribution of the fine and coarse
fractions obtained following air classification

| | Feeding rate | Fine Fraction (micron) | | | Coase Fraction (micron) | | |
|---|---|---|---|---|---|---|---|
| Variety | (kg/hr.) | D(50) | D(90) | D(4,3) | D(50) | D(90) | D(4,3) |
| Commodity Pea #2 | 2.3 | 6.9 | 15.9 | 8.5 | 21.1 | 33.6 | 21.9 |
| Commodity Pea #3 | 2.9 | 6.6 | 17.2 | 8.8 | 20.7 | 33.0 | 21.5 |
| Commodity Pea #4 | 3.0 | 6.0 | 15.6 | 7.8 | 20.2 | 32.0 | 20.9 |
| High Protein | 2.7 | 6.2 | 14.9 | 7.6 | 20.5 | 32.1 | 20.9 |
| Pea #2 | | | | | | | |

### Example 3: Chemical and Physical Analysis of Protein-Enriched Pea Compositions Protein Content Analysis

Protein content of a pea composition is measured using a combustion method (e.g., ISO 16634-2:2016). Briefly, the sample to be analyzed is combusted at high temperature in an oxygen atmosphere, any nitrogen found in the sample is then converted to N₂, which is detected and quantified using a thermal conductivity detector.

Protein content of a pea composition may alternatively be measured using the Kjeldahl method (Wybraniec S., et. al. An overview of the Kjeldahl method of nitrogen determination. Part II. Sample preparation, working scale, instrumental finish and quality control. Crit. Rev. Anal. Chem. 2013; 43:224-272.). First, the sample to be analyzed is heated to 360-410° C. and digested using concentrated sulfuric acid in the presence of a catalyst, such as selenium or copper. Next, the pH of the solution is raised using sodium hydroxide in order to convert any ammonium (NH₄⁺) in solution (derived from nitrogen in the digested sample) in to ammonia gas (NH₃), which is then distilled off into an aqueous HCl solution of known volume and concentration. This solution may then be titrated in order to determine its pH, and back-calculate the amount of nitrogen in the original sample. The approximate percent weight of protein in a sample is calculated by multiplying the percent weight of nitrogen in the original sample by a factor of 6.25.

Protein content of a pea, a batch of peas, or a pea composition may also be measured using near-infrared spectroscopy (NIRS). An overview of NIRS measurements is detailed in Zhu et al. Determination of soybean routine quality parameters using near-infrared spectroscopy. Food Sci. Nutr. 2018, 6, 1109-1118. Samples are placed in position relative to a light source and detector for an near-infrared spectrometer, such as a fourier-transform near-infrared spectrometer (FT-NIR) with a scanning spectral range of 3700-15,000/cm. Spectra are collected over the range 4000-12,600/cm, at a resolution of 16/cm with 60 scan number, which containing the absorbance regions of the traits of interest (4000-9000/cm for protein, moisture, and fat). Each sample is scanned three times to eliminate differences caused by objective factors. The spectra are compared to calibration or standard spectra, the protein content of which has been confirmed by other laboratory methods, such as combustion analysis or the Kjeldahl method.

### Crude Fiber Content Analysis

Fiber content of a pea composition is measured using standardized methods (AOAC (1990) method 985.29). Wherein duplicate test portions of the sample are digested with enzymes (i) a-amylase, (ii) protease, (iii) amyloglucosidase. The residue is allowed to precipitate and is filtered. One residue goes for protein analysis, the other for ash analysis. Total dietary fiber=weight of the residue−weight (ash+protein).

### Insoluble Dietary Fiber and Soluble Dietary Fiber

The total dietary fiber of a pea composition may be further analyzed according to any suitable methods known in the art, to determine its profile of different categories of fiber and starch therein, including insoluble dietary fiber and soluble fiber.

### Ash Content Analysis

Ash content of a pea composition is measured using standard methods. A sample of the pea composition is first dried. Then, the sample is weighed. The sample is then placed in a crucible, and placed in a high temperature muffle furnace for a period of 24 hours at 500 to 600° C. in order to dry ash the sample. Once the sample has been ashed, the remaining solids are weighed in order to determine the weight percent of ash in the sample.

### PDI Analysis

Protein dispersibility index (PDI) of a pea composition is measured as follows: 20 g of a pea sample is weighed and blended. 300 mL deionized water is measured and 50 mL of the deionized water is added to the blended pea sample. The deionized water and blended pea sample are stirred to form a paste. The remaining 250 mL of deionized water is added to the paste, and the remaining deionized water and paste are blended at 8500 rpm for 10 minutes. After 10 minutes, the blended mixture is centrifuged at 2700 rpm for 10 minutes. The supernatants from the centrifuged samples are collected and weighed. The protein content of the supernatant is determined relative to the protein content of the original pea sample, and the PDI is calculated as the percentage of the protein in the supernatant divided by the percentage in the original pea sample (a PDI of 100 indicates total solubility).

### Particle Size Analysis

Particle size distributions are measured using laser diffraction by measuring the angular variation in intensity of light scattered as a laser beam passes through a dispersed particulate sample. Large particles scatter light at small angles relative to the laser beam and small particles scatter light at large angles. The angular scattering intensity data is then analyzed to calculate the size of the particles responsible for creating the scattering pattern, using the Mie theory of light scattering. The particle size is reported as a volume equivalent sphere diameter.

### Example 4: Composition Analysis of Field Pea Cultivar 3997499

The chemical composition of a sample of peas produced from field pea cultivar 3997499 was analyzed. For comparison, a sample of peas produced from a commodity pea cultivar was also analyzed. The results of this analysis are provided in Table 5, below. Also provided in Table 5 are the standard methods used to analyze each component of the peas.

TABLE 5
Chemical composition of a commodity
pea and field pea cultivar 3997499

| | | Commodity | Pea Cultivar |
|---|---|---|---|
| Reference / Method | Parameter | Pea | 3997499 |
| Moisture-Forced Draft | Moisture - Forced | 11% | 11% |
| Oven: AOCS Ba 2a-38 | Daft Oven | | |
| Combustion: AOAC | Protein (DB) | 24% | 31% |
| 992.15; AOAC 990.03 | | | |
| AOAC 954.02 | Fat by acid | 2.1% | 2.0% |
| | hydrolysis (DB) | | |
| Soxhlet method or | Crude Fat/Oil | 1.1% | 2.3% |
| Crude Fat/Oil; AOCS | (DB) | | |
| Ac 3-44 | | | |
| AOAC 2009.01; | Soluble Fiber | 1.2% | 1.6% |
| AOAC 2011.25 | (DB) | | |
| AOAC 2009.01; | Insoluble Fiber | 18% | 18% |
| AOAC 2011.25 | (DB) | | |
| AOAC 991.43 | Total Dietary | 19% | 19% |
| | Fiber (DB) | | |
| AOAC 962.09; AOCS | Crude Fiber (DB) | 5.7% | 5.2% |
| Ba 6-84 | | | |
| AOAC 942.05 | Ash (DB) | 3.1% | 3.1% |
| AOAC 996.11 | Total Starch | 47% | 41% |

DB = Dry weight basis

Reference to “about” a value or parameter herein includes (and describes) embodiments that are directed to that value or parameter per se. For example, description referring to “about X” includes description of “X”. In some embodiments, the term “about” when used in association with a measurement, or used to modify a value, a unit, a constant, or a range of values, refers to variations of +/−2%.

Reference to “between” two values or parameters herein includes (and describes) embodiments that include those two values or parameters per se. For example, description referring to “between x and y” includes description of “x” and “y” per se.

### DEPOSIT INFORMATION

A deposit of the Benson Hill Seeds, Inc. proprietary field pea cultivar 3997496 disclosed above and recited in the appended claims is maintained by Benson Hill Seeds, Inc. A deposit will be made with Provasoli-Guillard National Center for Marine Algae and Microbiota, Bigelow Laboratory for Ocean Sciences (NCMA). Access to this deposit will be available during the pendency of this application to persons determined by the Commissioner of Patents and Trademarks to be entitled thereto under 37 C.F.R. 1.14 and 35 U.S.C. § 122. Upon allowance of any claims in this application, all restrictions on the availability to the public of the variety will be irrevocably removed by affording access to a deposit of at least 625 seeds of the same variety with NCMA. The deposit will be maintained in the depository for a period of 30 years, or 5 years after the last request, or for the effective life of the patent, whichever is longer, and will be replaced if necessary, during that period.

A deposit of the Benson Hill Seeds, Inc. proprietary field pea cultivar 3997499 disclosed above and recited in the claims herein is maintained by Benson Hill Seeds, Inc. A deposit will be made with Provasoli-Guillard National Center for Marine Algae and Microbiota, Bigelow Laboratory for Ocean Sciences (NCMA). Access to this deposit will be available during the pendency of this application to persons determined by the Commissioner of Patents and Trademarks to be entitled thereto under 37 C.F.R. 1.14 and 35 U.S.C. § 122. Upon allowance of any claims in this application, all restrictions on the availability to the public of the variety will be irrevocably removed by affording access to a deposit of at least 625 seeds of the same variety with NCMA. The deposit will be maintained in the depository for a period of 30 years, or 5 years after the last request, or for the effective life of the patent, whichever is longer, and will be replaced if necessary, during that period.

A deposit of the Benson Hill Seeds, Inc. proprietary field pea cultivar 3998902 disclosed above and recited in the claims herein is maintained by Benson Hill Seeds, Inc. A deposit will be made with Provasoli-Guillard National Center for Marine Algae and Microbiota, Bigelow Laboratory for Ocean Sciences (NCMA). Access to this deposit will be available during the pendency of this application to persons determined by the Commissioner of Patents and Trademarks to be entitled thereto under 37 C.F.R. 1.14 and 35 U.S.C. § 122. Upon allowance of any claims in this application, all restrictions on the availability to the public of the variety will be irrevocably removed by affording access to a deposit of at least 625 seeds of the same variety with NCMA. The deposit will be maintained in the depository for a period of 30 years, or 5 years after the last request, or for the effective life of the patent, whichever is longer, and will be replaced if necessary, during that period.

A deposit of the Benson Hill Seeds, Inc. proprietary field pea cultivar 4004764 disclosed above and recited in the claims herein is maintained by Benson Hill Seeds, Inc. A deposit will be made with Provasoli-Guillard National Center for Marine Algae and Microbiota, Bigelow Laboratory for Ocean Sciences (NCMA). Access to this deposit will be available during the pendency of this application to persons determined by the Commissioner of Patents and Trademarks to be entitled thereto under 37 C.F.R. 1.14 and 35 U.S.C. § 122. Upon allowance of any claims in this application, all restrictions on the availability to the public of the variety will be irrevocably removed by affording access to a deposit of at least 625 seeds of the same variety with NCMA. The deposit will be maintained in the depository for a period of 30 years, or 5 years after the last request, or for the effective life of the patent, whichever is longer, and will be replaced if necessary, during that period.

A deposit of the Benson Hill Seeds, Inc. proprietary field pea cultivar 4004781 disclosed above and recited in the claims herein is maintained by Benson Hill Seeds, Inc. A deposit will be made with Provasoli-Guillard National Center for Marine Algae and Microbiota, Bigelow Laboratory for Ocean Sciences (NCMA). Access to this deposit will be available during the pendency of this application to persons determined by the Commissioner of Patents and Trademarks to be entitled thereto under 37 C.F.R. 1.14 and 35 U.S.C. § 122. Upon allowance of any claims in this application, all restrictions on the availability to the public of the variety will be irrevocably removed by affording access to a deposit of at least 625 seeds of the same variety with NCMA. The deposit will be maintained in the depository for a period of 30 years, or 5 years after the last request, or for the effective life of the patent, whichever is longer, and will be replaced if necessary, during that period.

A deposit of the Benson Hill Seeds, Inc. proprietary field pea cultivar 4009968 disclosed above and recited in the claims herein is maintained by Benson Hill Seeds, Inc. A deposit will be made with Provasoli-Guillard National Center for Marine Algae and Microbiota, Bigelow Laboratory for Ocean Sciences (NCMA). Access to this deposit will be available during the pendency of this application to persons determined by the Commissioner of Patents and Trademarks to be entitled thereto under 37 C.F.R. 1.14 and 35 U.S.C. § 122. Upon allowance of any claims in this application, all restrictions on the availability to the public of the variety will be irrevocably removed by affording access to a deposit of at least 625 seeds of the same variety with NCMA. The deposit will be maintained in the depository for a period of 30 years, or 5 years after the last request, or for the effective life of the patent, whichever is longer, and will be replaced if necessary, during that period.

What is claimed is:

1. A method of producing a protein-enriched pea composition, the method comprising:
a) providing peas, wherein at least a portion of the peas has a protein content of at least about 25% pea proteins on a dry weight basis;
b) grinding the peas to provide a ground pea composition; and
c) isolating a protein-enriched pea composition from the ground pea composition,
wherein the protein-enriched pea composition comprises at least about 60% pea proteins on a dry weight basis,
wherein (i) the peas comprise peas of field pea cultivar 3997496, wherein a representative sample of seed of said cultivar is deposited under NMCA No. ______; or
(ii) the peas comprise peas of field pea cultivar 3997499, wherein a representative sample of seed of said cultivar is deposited under NMCA No. ______; or
(iii) the peas comprise peas of field pea cultivar 3998902, wherein a representative sample of seed of said cultivar is deposited under NMCA No. ______; or
(iv) the peas comprise peas of field pea cultivar 4004764, wherein a representative sample of seed of said cultivar is deposited under NMCA No. ______; or
(v) the peas comprise peas of field pea cultivar 4004781, wherein a representative sample of seed of said cultivar is deposited under NMCA No. ______; or
(vi) the peas comprise peas of field pea cultivar 4009968, wherein a representative sample of seed of said cultivar is deposited under NMCA No. ______,
or any combination of (i)-(vi).

2. A method of producing a protein-enriched pea composition, the method comprising:
a) providing peas, wherein at least a portion of the peas has a protein content of at least about 25% pea proteins on a dry weight basis;
b) grinding the peas to provide a ground pea composition; and
c) isolating a protein-enriched pea composition and a starch-enriched pea composition from the ground pea composition,
wherein the protein-enriched pea composition comprises at least about 60% pea proteins on a dry weight basis, between about 1% and 5% by weight crude fiber, and between about 5% and 10% by weight ash;
wherein the weight of the protein-enriched pea composition is at least about 20% of the weight of the ground pea composition;
wherein the protein-enriched pea composition has a protein dispersibility index of at least 75; and
wherein the starch-enriched pea composition comprises between about 10% and 25% by weight protein, between about 0.1% and 5% by weight crude fiber, between about 1% and 5% by weight ash, and between about 1% and 5% by weight damaged starch.

3. The method of claim 1 or 2, further comprising dehulling the peas prior to grinding.

4. The method of any one of claims 1-3, wherein the isolation of a protein-enriched pea composition in step c) is accomplished using an air classification process.

5. The method of any one of claims 1-4, wherein a) the weight % protein content of the protein-enriched pea composition, multiplied by b) the % yield of the protein-enriched pea composition, is at least 10%.

6. The method of any one of claims 1-5, wherein a single separation step is performed in the method.

7. The method of any one of claims 1-6, wherein the peas have an average starch content of less than about 45% starch on a dry weight basis.

8. The method of any one of claims 1-7, wherein the peas have an average protein to starch ratio of at least about 0.75.

9. The method of any one of claims 2-8, wherein the peas comprise peas of field pea cultivar 3997496, wherein a representative sample of seed of said cultivar is deposited under NMCA No. ______.

10. The method of any one of claims 2-8, wherein the peas comprise peas of field pea cultivar 3997499, wherein a representative sample of seed of said cultivar is deposited under NMCA No. ______.

11. The method of any one of claims 2-8, wherein the peas comprise peas of field pea cultivar 3998902, wherein a representative sample of seed of said cultivar is deposited under NMCA No. ______.

12. The method of any one of claims 2-8, wherein the peas comprise peas of field pea cultivar 4004764, wherein a representative sample of seed of said cultivar is deposited under NMCA No. ______.

13. The method of any one of claims 2-8, wherein the peas comprise peas of field pea cultivar 4004781, wherein a representative sample of seed of said cultivar is deposited under NMCA No. ______.

14. The method of any one of claims 2-8, wherein the peas comprise peas of field pea cultivar 4009968, wherein a representative sample of seed of said cultivar is deposited under NMCA No. ______.

15. A protein-enriched pea composition produced according to the method of any one of claims 1-14.

16. The composition of claim 15, wherein the composition is a flour.
